(19) United States
(12) Patent Application Publication
(10) Pub. No.: US 2003/0135876 A1
(43) Pub. Date: Jul. 17, 2003

(54) Maintenance of genetic lines using cryopreserved sperm

(76) Inventors: Larry Mobraaten, Bar Harbor, ME (US); Carlisle Landel, Southwest Harbor, ME (US)

Correspondence Address: HOWREY SIMON ARNOLD & WHITE, LLP, BOX 34, 301 RAVENSWOOD AVE., MENLO PARK, CA, 94025, US

(21) Appl. No.: 10/051,956

(22) Filed: Jan. 16, 2002

(51) Int. Cl. C12N 15/85; A01K 67/00

(52) U.S. Cl. 800/21; 435/455

(57) ABSTRACT

The present invention relates to a method for increasing the fertilization capability of cryopreserved sperm in in vitro fertilization. In particular, the invention provides methods and compositions for the manipulation of oocytes, which results in increased fertilization rates of the cryopreserved sperm. The invention allows for the maintenance of genetic lines with the use of cryopreserved stored sperm with minimal technical expertise and resources.

### FIELD OF THE INVENTION

The invention relates to the use of cryopreserved sperm for the maintenance of genetic lines in mammalian models. More specifically, the invention provides for methods of manipulating mammalian oocytes to increase the fertilizability of said cryopreserved sperm in order to maintain genetic lines. The invention may be applied to improve in vitro fertilization and other assisted fertilization means.

### BACKGROUND OF THE INVENTION

The number of mouse strains used in biomedical research is growing rapidly due to the ability to selectively alter mammalian genomes using transgenic and targeted mutation technology. As a result, large numbers of potentially important mutations are being created and propagated in new strains of mice. Producing each of these strains represents a significant outlay of resources. The vast majority of them have some immediate research utility, but after this initial period of research activity, they may have only a small amount of use, or may have no immediate use whatsoever. Future scientific discoveries, however, may render a strain useful again, necessitating the maintenance of all strains to ensure continuity and advancement in biomedical research. Thus, in the aggregate, all strains represent a resource of experimental material that is critical for the progress of biomedical research, and in an ideal world, all of these strains would be maintained in a way that would preserve this resource and insure against its loss.

Maintaining this resource through the maintenance of colonies of live mice is expensive. In particular, not enough space exists to house all these animals. Furthermore, in order to insure against losing a strain due to accident, a particular strain would ideally be housed in two separate sites, which effectively halves the available space. This in turn introduces a further complication because of genetic drift, i.e., the accumulation of random mutations that occurs naturally as a strain is propagated. Unless the separated lines of a single mouse strain are allowed to interbreed, they will drift apart genetically.

One cost-effective solution to this dilemma is to maintain mouse strains through cryopreservation. Cryopreservation of embryos and gametes assures the continuity of the resource, and assures the availability of a wide diversity of research materials for biomedical research in a cost effective manner. Cryopreservation of gametes circumvents the need for a large amount of storage space as well as a high level of technical expertise in order to preserve genetic material of experimental lines (Critser and Mobraaten, 2000). Cryopreservation is widely used in humans and other animal systems as a means of storing genetic material for propagating mammalian lines. Methods for embryo cryopreservation as a means to back up and preserve scientifically valuable strains of inbred mice are well established and the value of this strategy has been amply demonstrated (Glenister et al., 1990; Glenister and Thornton, 2000; Mobraaten, 1986; Mobraaten, 1998; Mobraaten, 1999; Sharp and Mobraaten, 1997; and Sharp et al., 1997). Frozen embryos are also increasingly being used as a means to introduce and re-derive strains of mice into barrier, or germ-free facilities (Baker, 1988; Morrell, 1999; Reetz et al., 1988; Suzuki et al., 1996).

Although frozen embryos are routinely used to rescue unique genetic lines of experimental animals, this method is prohibitive for many practitioners due to its high technological complexity. In addition, the viability and survivability characteristics of mouse embryos dictates that a large number (approximately 100-500) of frozen embryos is typically frozen in order to assure the successful reestablishment of a breeding colony from frozen stock (Nakagata et al, 1997). These embryos are obtained either from the oviducts of mated females or through in vitro fertilization and culturing of embryos prior to freezing. Therefore, a breeding colony of a particular genetic line, prior to reducing or abolishing it, must be expanded at considerable effort in order to maintain it in a frozen state.

Cryopreservation of mouse sperm, in contrast to embryos, is relatively simple, and requires few resources and a relatively low level of technical expertise (Sztein et al, 1997; Sztein et al., 2000b; Sztein et al., 2001). Sperm cryopreservation also has the advantage that a large amount of material can be easily isolated. For example, a single fertile male usually provides on the order of 10⁷ sperm, providing enough material to generate very large numbers of mice, even if the efficiency with which they are produced is fairly low. This circumvents the need for the availability of a multitude of breeder males and females necessary in conventional breeding scenarios or with the use of frozen embryos.

Despite the advantages that cryopreservation of sperm may play in the maintenance of genetic lines, cryopreservation of mouse sperm has been hampered by the fact that sperm from inbred strains of mice fail to fertilize after cryopreservation and thawing (Nakagata et al, 1990; Sztein et al., 2000a). Although successful recovery of live pups from frozen sperm has been reported in the literature since 1990 (Yokoyama et al., 1990), and improved methods for sperm cryopreservation have been developed since that time (Nakagata et al., 1992 and Sztein et al, 1997), the techniques give extremely poor results with sperm from inbred strains of mice.

The failure of cryopreserved inbred mice sperm to fertilize ova has been partially attributed to the emergence of defective acrosomal structures after cryopreservation. Acrosomes upon thawing appear damaged or reacted after cryopreservation in scanning electron micrographs and after biochemical staining analyses. During normal fertilization events, when the sperm reaches the egg in the oviduct, the enzymes in acrosomal structures contained in the head of the sperm allow the sperm to penetrate the zona pellucida, the glycoprotein “shell” that surrounds and protects the egg and, after fertilization, the pre-implantation embryo. In the absence of acrosomal enzymes and general acrosomal function, the sperm cannot penetrate the zona pellucida, resulting in failed fertilization and lack of zygote formation. In addition to defective acrosomal structures, cryopreserved sperm may also possess decreased motility, further contributing to the low or non-existent fertilization rates.

At the present time, there are only a few methods that have been published that circumvent this block to fertilization resulting from cryopreservation and thawing. Nakagata et al. (1997) disclosed a method of partial zona dissection, in which the zona pellucida is physically nicked with a hand-held needle. The nick produces an open channel through the zona pellucida, allowing the sperm free, unobstructed access to the oocyte. Intracytoplasmic sperm injection (ICSI) has also been described, wherein sperm is injected directly into the oocyte (Kimura and Yanagimachi, 1995). Although live birth of embryos have been reported using these techniques, significant problems exist which prevent the widespread use of these technologies. In particular, partial zona dissection is laborious and not efficient, while intracytoplasmic sperm injection requires tremendous skill and resources. That partial zona dissection and intracytoplasmic sperm injection overcome the fertilization block of cryopreserved sperm is evidence that defective acrosomal structures, or structures which assist in the penetration of the zona pellucida to the oocyte membrane, may be responsible for decreased or non-existent in vitro fertilization events. Studies in humans and other species also suggest that zona manipulation is helpful in overcoming the lack of successful fertilization caused by defective sperm (Odawara et al., 1993 and Odawara et al., 1995).

In humans, zona manipulation or removal is also performed to increase implantation rates of embryos created by in vitro fertilization. Immediately prior to normal implantation, the zona pellucida, through the combined action of alternate contraction and expansion of the developing blastocyst as well as by lytic substances released by the embryo and/or uterus, dissolves. Assisted hatching, or release of the blastocyst, through zona removal or directed circumferate thinning has been shown to increase implantation rates and subsequent pregnancies after in vitro fertilization and culturing to the blastocyst stage in humans, mice and other species (Khalifa et al., 1992; Gordon and Dapunt, 1993 and Mansour et al., 2000). Although successful fertilization and implantation has occurred in the absence of the zona pellucida, the zona during normal fertilization plays an important role in the protection of the developing embryo as well as a block to polyspermy. After entrance of the first sperm into the oocyte, a process known as zona hardening takes place whereby the zona pellucida undergoes a biochemical change which blocks the entrance and/or attachment of additional sperm to the oocyte. The incidence of polyspermy does increase in the absence of a functional zona pellucida (Odawara et al., 1995).

Although the zona pellucida is thought to play an important role in the protection of the developing embryo as well as a block to polyspermy, removal of the zona pellucida to increase successful zygote formation in in vitro fertilization or assisted hatching has been reported to have a minimal effect on the production of normal mice from embryos developed in the absence of a zona pellucida. Naito et al. (1992) report that the production of normal mice was only slightly inferior to that of zona-intact embryos in in vitro fertilization. The survival rate of zona-free embryos in assisted hatching are also comparable to that of normal embryos, and in fact improve the outcome of poor prognosis patients when used in conjunction with in vitro fertilization (Mansour et al., 2000).

### SUMMARY OF THE INVENTION

The invention provides for methods and compositions which improve the fertilization rate of cryopreserved sperm and allows for the maintenance of genetic lines through cryopreserved gametes.

It is an object of the present invention to provide methods and compositions for the maintenance of genetic lines in mammals. In particular, it is an object of the invention to maintain genetic lines through the use of cryopreserved sperm. Cryopreservation of sperm allows the maintenance and storage of an ample supply of genetically useful mammalian lines.

The invention provides methods for the maintenance of genetic lines in a non-human mammal comprising: a. isolating oocytes from a donor female of a non-human mammal to produce isolated oocytes; b. reducing the circumferential thickness of the zona pellucida layer surrounding the isolated oocytes without removing the zona pellucida layer completely; c. fertilizing the isolated oocytes in vitro with cryogenically preserved sperm from the same species as the non-human mammal above to produce at least one fertilized embryo; and d. transplanting the fertilized embryo to a recipient female of the same non-human mammal for implantation and placental development.

The invention also provides for methods for the fertilization of a non-human mammalian oocyte using cryopreserved sperm. In particular, the invention provides for methods to increase the fertilization capacity of cryopreserved sperm through the manipulation of the mammalian oocyte. Specifically, the invention provides for a method of manipulating the zona pellucida of a non-human mammalian oocyte in order to increase the fertilization capability of cryopreserved sperm such that the circumferential thickness of the zona pellucida layer surrounding the non-human mammalian oocyte is decreased, while maintaining an intact zona pellucida layer, comprising removing cumulus cells surrounding the oocyte and treating the oocyte with an agent to reduce the cirumferential thickness of the zona pellucida layer. Cryopreserved sperm are often defective in either acrosomal content or other reproductive functions, making them unable to penetrate the protective zona pellucida membrane surrounding the mature oocyte.

The invention overcomes the block to fertilization of cryopreserved sperm by manipulating the zona pellucida of recipient mammalian oocytes such that cryopreserved sperm, which are deficient or defective, can penetrate the surrounding zona pellucida to initiate fertilization. The methods disclosed in the invention allow the manipulation of the zona pellucida such that cryopreserved sperm may inseminate oocytes in vitro, yet still maintain the integrity of the zona pellucida to allow for protection of the developing embryo. The invention provides for a simplified means to manipulate the zona pellucida without the need for a large amount of technical expertise. The invention also provides for the rapid processing of a large number of oocytes for zona manipulation and subsequent in vitro fertilization.

Another aspect of the invention relates to an isolated oocyte wherein the circumferential thickness of the zona pellucida layer surrounding the isolated oocyte is reduced. The isolated oocyte is used in in vitro fertilization to increase the fertilization capability of cryopreserved or otherwise defective sperm.

Yet another aspect of the invention relates to the use of the methods disclosed herein in other species which require maintenance of genetic lines for experimental or other purposes. Yet another aspect of the invention relates to the use of the methods described for any species through which manipulation of the zona pellucida is necessary to overcome a block to fertilization from defective or otherwise deficient sperm.

### BRIEF DESCRIPTION OF THE DRAWINGS

A more complete appreciation of the invention will be readily obtained by reference to the following Detailed Description of the Invention and the accompanying drawings, wherein:

FIG. 1 outlines the events of zona melting for assisted in vitro fertilization of cryopreserved sperm, whereby PBS=phosphate buffered saline, BSA=bovine serum albumin, HTF=human tubal fluid medium, IVF=in vitro fertilization.

FIG. 2 outlines results of the use of fresh (hatched bars) and cryopreserved (open bars) sperm from Fl and inbred mouse strains in in vitro fertilization.

FIG. 3 shows the results of the use of cryopreserved sperm from F1 (strains 3873 & 3874) and inbred mouse strains in in vitro fertilization of oocytes from which the zona pellucida has been removed.

FIG. 4 outlines the events of partial zona dissection in assisted in vitro fertilization of cryopreserved sperm, whereby HTF=human tubal fluid medium and IVF=in vitro fertilization.

FIG. 5 outlines the results of experiments comparing the fertilization rate of untreated (None) and zona melted (ZM) oocytes using frozen and fresh sperm from the inbred strain C57B1/6J. This box chart shows the range, mean, and 95% range for the data.

FIG. 6 shows the results of the use of cryopreserved sperm from various inbred mouse strains (strain) with no treatment, zona melting (Zona Melt) or laser nicking (Laser Nick) with in vitro fertilization.

FIG. 7 shows the results of the use of cryopreserved sperm from 48 mouse strains with no treatment (NT; open bars) and zona melting (ZM; hatched bars) in in vitro fertilization.

### DETAILED DESCRIPTION OF THE INVENTION

The methods disclosed herein provide for the fertilization of non-human mammalian oocytes in vitro using cryopreserved sperm. Preferably, the non-human mammal is a mouse. However, a non-human mammal may be a rat, a hamster, a guinea pig, a horse, a pig, a goat, a sheep, a primate or any other non-human mammal whereby isolated sperm used in fertilization is defective in penetrating the zona pellucida layer for fertilization of the oocyte.

A preferred embodiment of the invention provides for the use of zona-manipulated oocytes to overcome the block to fertilization of cryopreserved sperm. In particular, cryopreserved sperm from inbred mice strains exhibit defective acrosomal structure and function, as well as low motility, contributing to the inability of cryopreserved sperm from inbred mice strains to fertilize oocytes in vitro. Although the non-human mammalian sperm may be rendered defective through a cryopreservation process as disclosed herein, one of ordinary skill in the art will recognize sperm may be rendered defective through other means which make it difficult or impossible for the isolated sperm to fertilize an isolated oocyte in vitro. The present invention is therefore intended to compensate for any isolated sperm defective in fertilizing isolated oocytes in vitro due to an inability to penetrate the zona pellucida layer for fertilization of the oocyte.

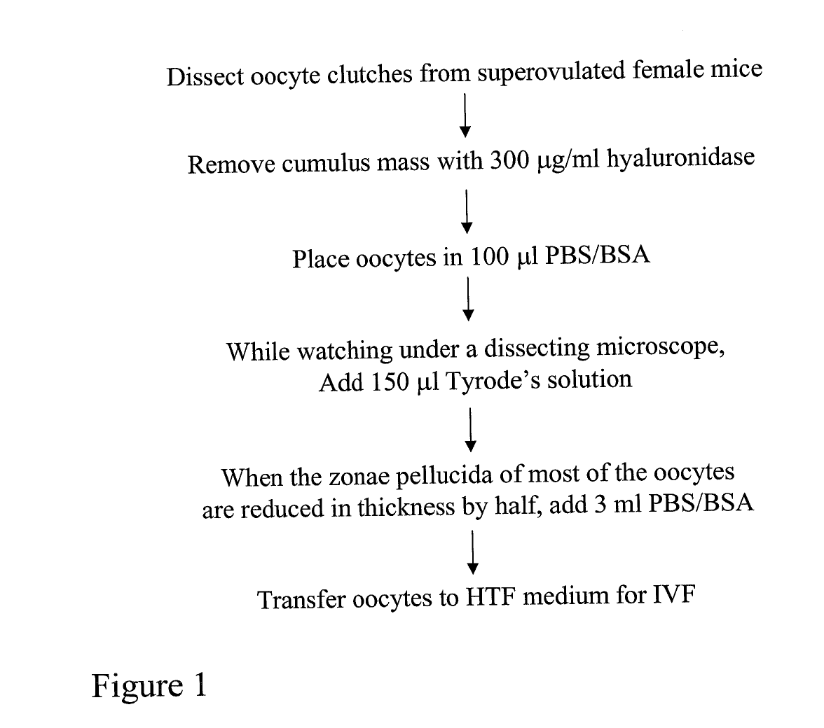

Zona-manipulation of oocytes may be carried out in a variety of ways. A preferred embodiment, shown in FIG. 1, includes the use of Acid Tyrode's solution to melt or decrease the circumferential thickness of the zona pellucida layer surrounding the mammalian oocyte. The circumferential thickness is the thickness of the zona pellucida layer surrounding the oocyte. Oocytes collected from superovulated females and freed from the cumulus cell mass by treatment with hyaluronidase are placed in Dulbecco's Modified Phosphate Buffered Saline supplemented with bovine serum albumin (PBS/BSA) medium. Acid Tyrode's medium is added dropwise to the oocytes. The oocytes are then observed using a dissecting microscope until approximately half the thickness of the zonae on the majority of the oocytes is dissolved. Because the zona on each oocyte dissolves at a different rate, this point is often reached when the first oocyte loses its zona. At this point the drop of oocytes is immediately flooded with an excess of PBS/BSA in order to neutralize the acid. Additional PBS/BSA is added and the oocytes with approximately half the thickness of the zonae remaining are used for in vitro fertilization.

Although the level of zona melting may vary from batch to batch of oocytes, the acidification of the oocytes disclosed herein sufficiently guide one of ordinary skill in the art to reach the endpoint value of removing approximately one-half of the circumferential layer of zona pellucida surrounding the oocyte. This event is observed under a dissecting microscope, wherein the melting of the zona pellucida is readily observed and visible. The rate of degradation may also vary from batch to batch, depending upon the thickness of the zona pellucida, or upon the efficiency of hyaluronidase digestion of the corona radiata surrounding the zona pellucida prior to zona manipulation. The rate of degradation may also vary dependent upon the amount of Acid Tyrode's added, the buffering capacity of the PBS/BSA and the pH of the PBS/BSA/Acid Tyrode's solution. A practitioner of ordinary skill in the art will recognize that a variety of buffer solutions and concentrations may be used in conjunction with the present invention to achieve zona melting of oocytes. However, it is important to note that endpoints must be closely monitored in order to avoid complete zonal removal. In addition, only oocytes with approximately one-half of the zona pellucida removed may be used in in vitro fertilization with cryopreserved sperm.

An alternative embodiment may be to decrease the pH of the medium containing the oocytes using other balanced salt solutions containing acid in order to melt the zonae pellucida such that only one-half the thickness of the zona pellucida is removed before in vitro fertilization. For example, the pH could be lowered by adding PBS (phosphate buffered saline), HTF (human tubal fluid medium) or normal saline solution that has been acidified to pH 2.5 with HCl. Longer incubation or degradation times may be required in order to achieve sufficient melting of the zona pellucida for effective fertilization. It will be appreciated by those of ordinary skill in the art that endpoints must be closely monitored to assure the removal of only approximately one-half the thickness of the zonae pellucida.

Other alternative embodiments for the partial removal of the zona pellucida include the use of other zona pellucida removing agents which may also melt the zona pellucida layer in a controlled fashion. Those of ordinary skill in the art recognize that there are many agents described which can melt, or reduce the circumferential thickness of the zona pellucida layer. These agents include pronase, trypsin and other enzymes or chemical compounds which have the effect of melting the zona pellucida surrounding the mammalian oocyte. Other agents may include acid variants of other balanced salt solutions that are used in conjunction with oocyte manipulation, in vitro fertilization or cell culturing. In addition, a mechanical means of removing approximately one-half the thickness of the zona pellucida in a controlled manner, such as by laser, needle or other mechanical manipulation of the zona pellucida, may also be used. Protocols must be adapted to allow the removal of the zona pellucida such that only approximately one half of the circumferential layer surrounding the oocyte is removed in a controlled fashion.

It is important that the zona pellucida is not completely removed during the zona melting step. Removing the zona pellucida requires the culturing of embryos to the blastocyst stage before surgical transfer to the uteri of recipient foster mothers if viable pups are to be obtained; further, each embryo must be cultured separately to avoid embryo-embryo fusion in culture. Also, embryos obtained from the in vitro fertilization of oocytes from which the zonae pellucida have been removed have a severely decreased rate of survival to parturition after transfer to foster mothers when compared to embryos with zonae pellucida. This is in direct contrast to the prior art, which discloses that oocytes with their zona pellucida removed develop at comparable rates to non-removed zona pellucida (Naito et al., 1992).

That the zona pellucida is left intact is also in contrast to prior studies which suggest that a pathway to the oocyte membrane is necessary due to the abnormalities in acrosomal structure and function. Prior art which successfully demonstrate a removal of the block to fertilization by cryopreserved sperm all require the creation of a clear pathway to the oocyte membrane. Techniques which remove the zona pellucida, or drill holes in the zona pellucida layer, include laser drilling, manual dissection, directed acid degradation or other methodologies whose commonality include the creation of a direct pathway to the oocyte membrane. To increase the percentage of fertilization events in in vitro fertilization, female mice may be superovulated using a treatment regimen of PMSG (pregnant mare serum gonadotropins) followed by hCG (human chorionic gonadotropin). The mice are first injected intraperitoneally with PMSG to stimulate follicular development in multiple follicles. The PMSG treatment mimics the action of FSH (follicle stimulating hormone), which stimulates select follicles for maturation. The mice are then injected approximately 48 hours later with hCG to stimulate further follicular development and induce ovulation of the multiple follicles previously stimulated by PMSG. Alternative embodiments include the use of human menopausal gonadotropin (hMG), which may increase egg yields over conventional PMSG/hCG treatment (Edirisinghe, W. R. et al., 1986) or other gonadotropins which mimic the actions of FSH and LH (lutenizing hormone) in inducing follicular development and ovulation of multiple oocytes.

The invention having been generally described, the following non-limiting examples are set forth as further illustrations of the invention. It is to be understood that the following examples are not meant to impose limitations upon the scope of the invention. On the contrary, it is to be clearly understood that one of ordinary skill in the art may refer to various other embodiments, modifications and equivalents thereof without departing from the spirit of the present invention and/or the scope of the appended claims.

### EXAMPLE 1

Cryopreservation and Thawing of Sperm

Sperm was recovered from mature male mice by removing the epididymides and vas deferentia and placing them in a culture dish containing 1 ml of 0.45 μm filtered-cryoprotectant (18% (D+) Raffinose pentahydrate (Sigma Chemical Co.), 3% Dehydrated Skim Milk (Difco, Inc.) in culture grade water) pre-warmed to 37° C. The organs were sliced 3 to 5 times with the edge of a hypodermic syringe needle and incubated in the culture dish for approximately 10 to 15 minutes to allow sperm to swim out of the organs. The samples were dispensed into 1.8 cc cryotubes (Nunc, Inc.) in 100 μl aliquots and exposed to liquid nitrogen vapors (approximately −120° C. for 10 minutes (cooling rate of approximately 20-40° C. per minute), then stored under liquid nitrogen.

Frozen samples of sperm were thawed by rapidly removing vials from liquid nitrogen storage and placing them into a water bath at 37° C. until all ice crystals are melted (approximately 2 minutes). The vial contents were gently mixed by tapping the tube lightly and the contents added directly to the IVF.

### EXAMPLE 2

Superovulation of Female Mice and Collection of Oocytes

19-23 day old female mice were injected intraperitoneally with 2.5 or 5.0 IU (international units) PMSG (pregnant mare serum gonadotropin; Sigma Chemical, Cat. # G-4877). This is followed by a 2.5 IU intraperitoneal injection of hCG (human chorionic gonadotropin; Sigma Chemical, Cat # CG-10) approximately 48 hours later. Approximately 13 hours later, females were sacrificed, starting with those injected earliest with hCG. The oviducts were dissected and placed in a drop of HTF medium (Quinn et al., 1985: 101.6 mM NaCl, 4.69 mM KCl, 0.20 mM MgSO₄.7H₂O, 0.37 mM KH₂PO₄, 2.04 mM CaCl₂.2H₂O, 25 mM NaHCO₃, 2.78 mM glucose, 0.33 mM Na pyruvate, 21.4 mM Na lactate, 0.075% penicillin-G, 0.05% streptomycin sulfate, 0.001% phenol red, 0.4%BSA; the pH is adjusted by gassing with 5% CO₂, 5% O₂ and 90% N₂). The ampullae were torn to release the egg clutches, and the clutches transferred to a single fertilization dish using a wide bore pipette tip. The process was repeated until all eggs were collected and distributed to petri dishes containing sperm from all male donors.

### EXAMPLE 3

In Vitro Fertilization and Blastocyst Culturing

Oocytes were collected from superovulated females 13 hours post hCG injection and the egg clutches were then transferred in as small a volume as possible to 0.5 ml HTF medium under an atmosphere of 5% CO₂, 5% O₂ and 90% N₂ at 37° C. containing 10⁵-10⁶ sperm. The sperm and eggs were incubated for approximately 4-6 hours. The fertilized eggs were then transferred through drops of fresh HTF, taking care to leave behind cumulus cells, sperm and debris. The embryos were then cultured overnight to the 2-cell stage. Embryos can be surgically transferred directly to the uteri of pseudopregnant foster mothers at this point using standard techniques (Hogan et al., 1994) or, if embryos of a later developmental stage were necessary or desired, the embryos can be transferred to KSOM medium (Lewitts and Biggers, 1991) and cultured to the proper stage.

### EXAMPLE 4

In Vitro Fertilization using Fresh and Cryopreserved Sperm

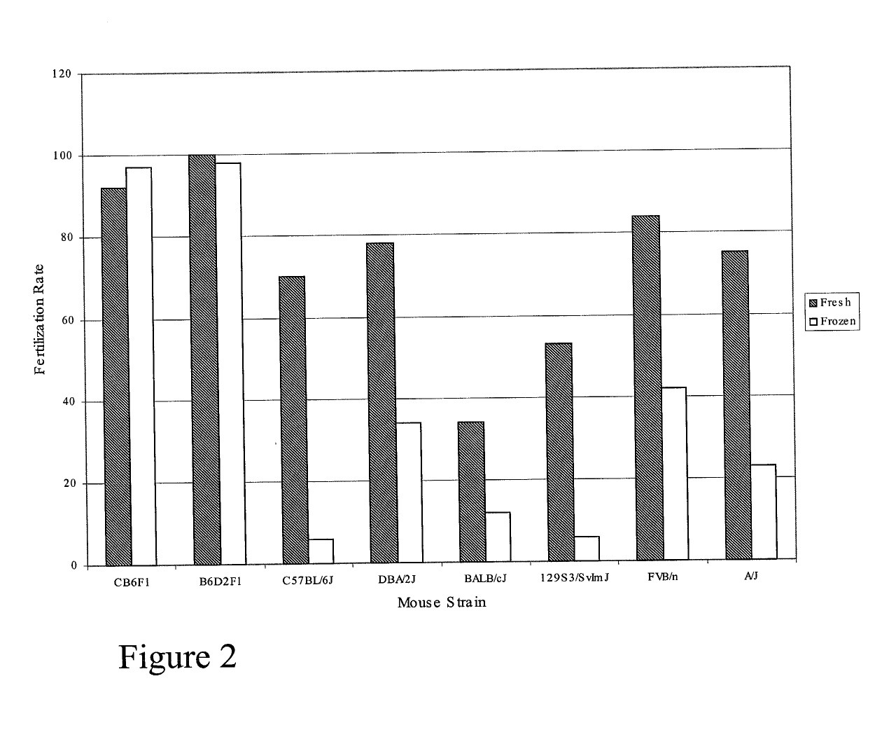

Fertilization rates in in vitro fertilization were measured in experiments using fresh and cryopreserved sperm from both F1 and inbred mouse strains. Oocytes were collected from superovulated females 13 hours post-hCG injection as described above, and the egg clutches were transferred in as small a volume as possible to 0.5 ml HTF media under an atmosphere of 5% CO₂, 5% O₂ and 90% N₂ at 37° C. containing 4-6×10⁶ fresh or cryopreserved sperm, and cultured for approximately 4-6 hours. The resulting two-cell embryos were scored and assayed for percent fertilization from each group. The results are shown in FIG. 2. The fertilization rate was consistently high for F1 (CB6F1 and B6D2F1) strains using either fresh or cryopreserved (frozen) sperm. However, fertilization rates varied for inbred mouse strains (C57BL/6J, DBA/2J, BALB/cJ, 129S3/SvInJ, FVB/n and AJ) using fresh sperm, and was consistently lower when in vitro fertilization was performed with cryopreserved sperm. These results agree with previous observations (Nakagata et al., 1992) and demonstrate the difficulty of performing in vitro fertilization using cryopreserved sperm in inbred mouse strains.

### EXAMPLE 5

In Vitro Fertilization of Zona-Free Oocytes with Cryopreserved Sperm

Fertilization rates with in vitro fertilization were measured using zona-free oocytes and cryopreserved sperm from both F1 and inbred mouse strains. Previous results suggested that removal of the zona pellucida only slightly impacted the production of normal mice in in vitro fertilization (Naito et al., 1992). Cumulus cells were removed as follows, so that the dissolution of the zonae could be observed. Oocytes were collected from superovulated females 13 hours post-HCG injection and placed in 500 μl phosphate buffered saline/BSA. The eggs were transferred to a sterile petri dish containing 300 μg/ml hyaluronidase (Type IV-S from bovine testes, Sigma) in PBS-BSA (Modified Dulbecco's PBS containing dextrose, pyruvate, Penicillin, Streptomycin and BSA: 0.8% NaCl, 0.02% KCl, 0.02% KH₂PO₄, 1.15% Na₂HPO₄, 0.01% MgCl₂.6H₂O, 0.1% CaCl₂, 0.1% dextrose, 3.6 mg sodium pyruvate, 0.075% penicillin-G, 0.05% streptomycin sulfate, 0.001% phenol red, 0.3% BSA) and gently pipetted up and down to help break up the clutches and corona radiata surrounding the individual oocytes. Once they were free of adhering cumulus cells, the oocytes were transferred through a succession of clean drops of PBS/BSA to separate them from the cumulus cells and then counted. The zonae pellucida of these eggs were removed by transferring them to a drop of acid Tyrode's solution (0.8% NaCl, 0.02% KCl, 0.01% MgCl₂.6H₂O, 0.1% CaCl₂.2H₂O, 0.1% dextrose, 0.4% polyvinylpyrrolidone, pH 2.5). Once the zonae had dissolved, the eggs were washed with PBS/BSA.

The clean eggs were then used for in vitro fertilization as follows. The eggs were transferred in as small a volume as possible to 0.5 ml HTF medium under an atmosphere of 5% CO₂, 5% O₂ and 90% N₂ at 37° C. containing approximately 4×10⁶ sperm. The eggs and sperm were incubated for 4 to 6 hours with the sperm at 37° C. under the mixed gas. The fertilized eggs were then washed by transferring the eggs from the in vitro fertilization solution to fresh HTF, taking care to leave behind as much debris as possible. The embryos were individually cultured overnight to the 2-cell stage and then transferred to individual microdrops of KSOM medium (Lawitts and Biggers, 1994) for further culture to the blastocyst stage. Blastocysts were then surgically transferred to the uteri of pseudopregnant foster mothers using standard techniques (Hogan, 1994).

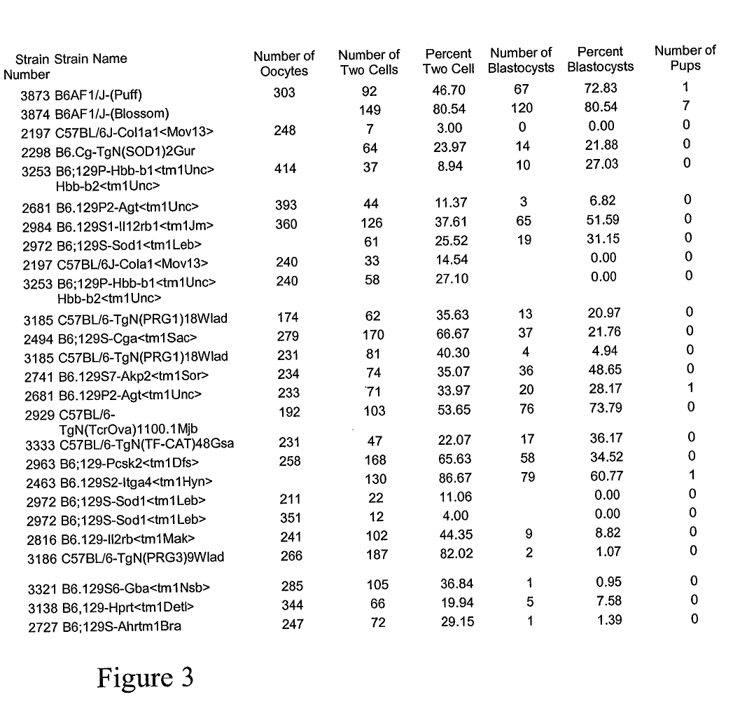

The results are shown in FIG. 3. Pups were obtained at a very low frequency when eggs without a zona pellucida are used for in vitro fertilization using cryopreserved sperm for all strains used. Only Strain 3874 (B6AF1/J-(Blossom)), which has an Fl genetic background and is not inbred, was capable of producing more than a single pup. These results emphasize the difficulty of working with zona-free oocytes, in contrast to previous results suggesting that zona-free embryos developed at comparable rates to intact zona pellucida oocytes (Naito et al., 1992).

### EXAMPLE 6

Zona Dissection of Isolated Oocytes

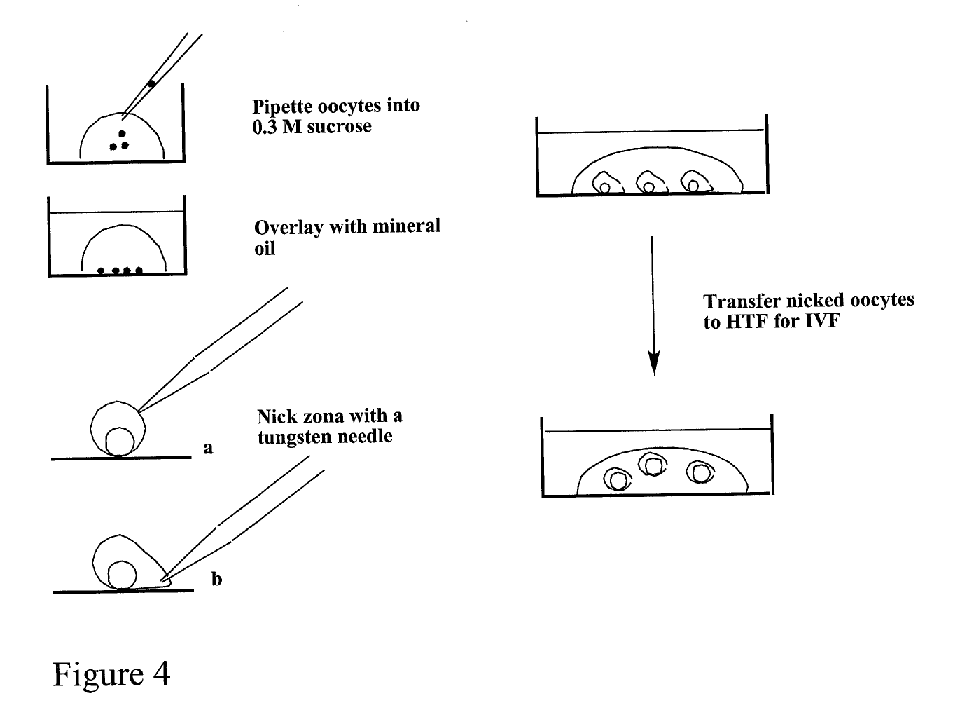

Partial zona dissection (FIG. 4) or laser nicking of isolated oocytes was performed to increase fertilization rates with cryopreserved sperm. Oocytes freed of cumulus cell mass with hyaluronidase treatment were washed and placed under a microscope. For partial zona dissection, the oocytes were pipetted into 0.3 M sucrose and overlaid with mineral oil to increase the viscosity of the solution and stabilize the oocytes. The oocytes were then nicked with a tungsten needle to open a channel to the oocyte membrane through the zona pellucida. Laser nicking also produces a channel through the zona pellucida to the oocyte membrane. In laser nicking, a hole in the zona pellucida is drilled using an infrared laser mounted on the dissecting microscope (Zona Laser Treatment System, Hamilton Thorne Research Laboratories). Zona drilled oocytes were washed in fresh PBS/BSA and used in in vitro fertilization.

### EXAMPLE 7

Zona Melting of Isolated Oocytes

Egg clutches from 12 sacrificed females were removed and placed in 500 μl phosphate buffered saline/BSA. The eggs were transferred to a sterile petri dish and and freed from the cumulus cells using hyaluronidase as described above.

Approximately half of the oocytes were then placed in a 100 μl drop of PBS/BSA in a sterile petri dish. While observing the eggs under a dissecting microscope, 150 μl of Acid Tyrode's was added to the oocyte suspension and the eggs were observed until the zonae of most of them had been reduced in thickness by about half. This melting of the zone takes place at a rate that varies from egg-to-egg, so that a convenient measure of the proper endpoint is when the first egg loses its zona. The oocytes are then quickly flushed with a 1 ml excess of PBS. An additional 2 ml of PBS is added to the petri dish, and the oocytes collected and transferred to HTF medium for IVF.

### EXAMPLE 8

Comparison of IVF Rates for Fresh and Frozen Sperm using Untreated and Zona Nicked Oocytes

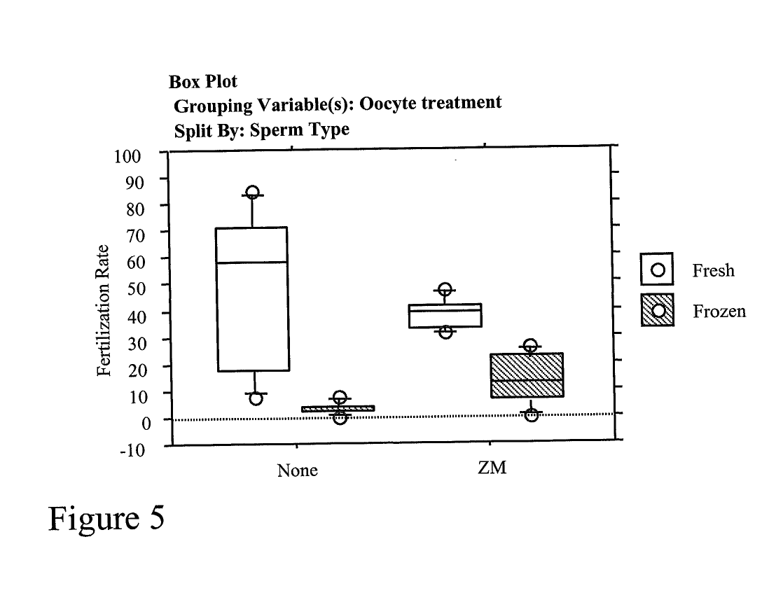

Oocytes were collected from superovulated C57B1/6 (B6) females as described above. Approximately half were treated with hyaluronidase and zona melted as described above. The other half was left untreated. The treated and untreated groups were then divided in half and fertilized in vitro with either fresh or frozen B6 sperm as described above, so that for each experiment there were four groups of IVFs: Fresh sperm with untreated oocytes, fresh sperm with zona-melted oocytes, frozen sperm with untreated oocytes, and frozen sperm with zona-melted oocytes. For each group, the fertilization rate was calculated. The experiment was repeated 7 times, and the data were pooled and analyzed using a t-test. The results are shown in FIG. 5. Zona melting of isolated oocytes significantly (p<0.001) improves the in vitro fertilization rate of frozen B6 sperm.

### EXAMPLE 9

Comparison of In Vitro Fertilization Rates with Zona Melting and Laser Nicking

The ability to recover pups from sperm from inbred strains using partial zona dissection or intracytoplasmic injection suggests that at least part of the block is the result of an inability of the sperm to penetrate the zona pellucida. However, both partial zona dissection and intracytoplasmic injection is laborious, time-consuming and requires high technical expertise to recover enough successfully fertilized embryos for either re-derivation or genetic line rescue experiments. Partial Zona dissection, in particular, is technically demanding, with a high loss of embryos due to physical damage and a strong probability of losing the zona from embryos during culture to the blastocyst stage. Because of the limitations of these previously published methods, other avenues were pursued to decrease the amount of time required to perform zona manipulation of the donor oocytes.

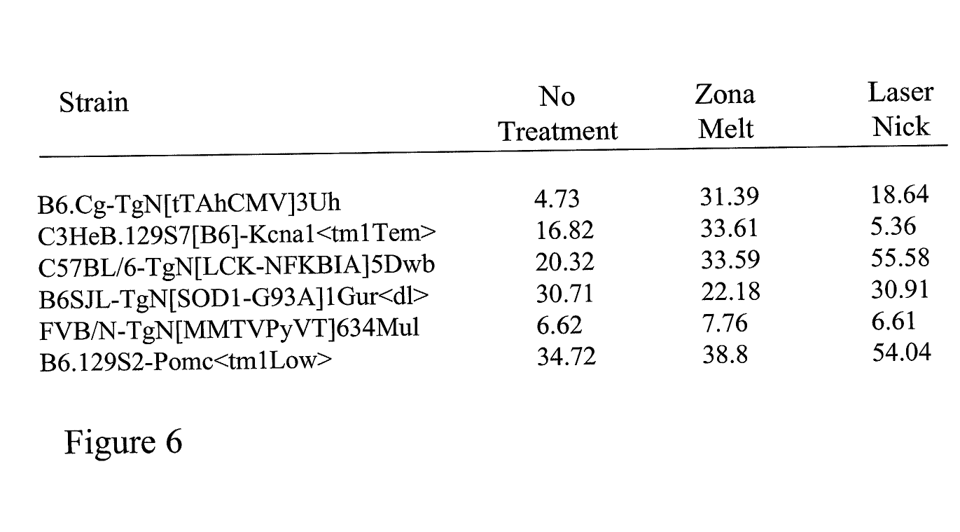

Previous studies to increase the fertilization rate of infertile or immobile sperm have focused on the manipulation of the zona pellucida to create holes or passageways into the oocyte through the zona layer. Laser nicking, a methodology that uses an infrared laser mounted on a microscope can be used to drill holes in the zona layer. Fertilization rates are dramatically improved, as seen in FIG. 6 depicting an increase in the formation of two-cell embryos with laser treatment (Laser Nick). Laser nicking, however, requires high technical expertise combined with large resources to perform such experiments.

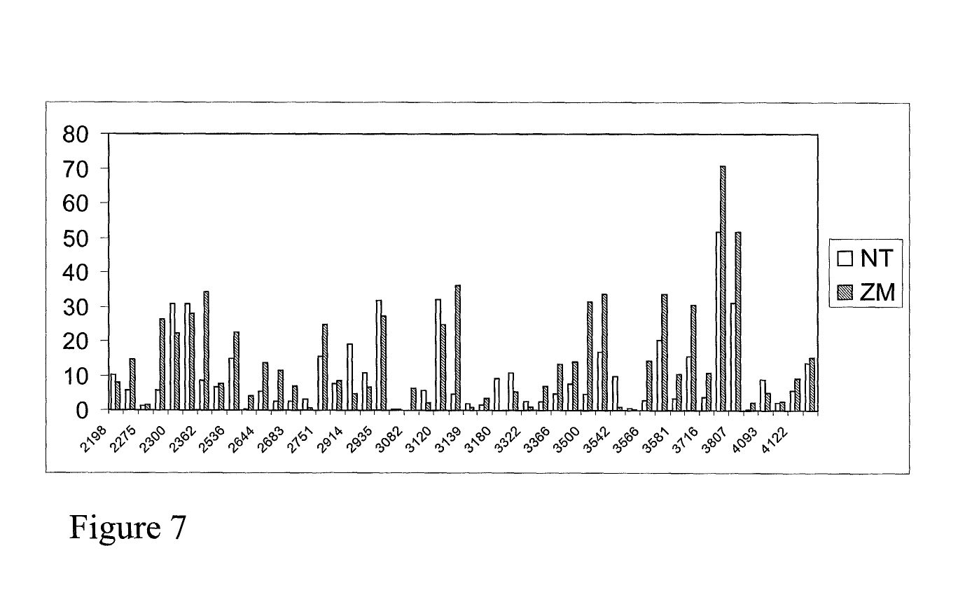

Although studies have indicated that a complete traversal of the zona pellucida was the only means to overcome the block to fertilization from cryopreserved sperm, problems associated with zonae-free embryos suggested otherwise. This prompted the manipulation of the protocol to maintain the integrity of the zona pellucida while allowing successful fertilization by cryopreserved sperm. Zona melting, which decreases the circumferential thickness of the zona pellucida to approximately one-half of the original thickness, also increases fertilization rates when cryopreserved sperm from inbred mouse strains are used (FIGS. 6 and 7). As compared to laser nicking, zona melting is comparably successful, increasing fertilization rates over oocytes that were left untreated (compare No Treatment vs. Zona Melt in FIG. 6). FIG. 7 expands this study, and shows that approximately 70% of all inbred mouse strains studied so far show an improvement in fertilization rates over oocytes that have been left untreated in in vitro fertilization. Approximately 30% of the treatments have no effect. In these cases, the lack of fertilization may be attributed to damage during the cryopreservation process that must affect other parts of the fertilization process, or that the oocytes are damaged during the zona manipulation process.

### REFERENCES

Baker, H. J. (1988) Rederivation of inbred strains of mice by means of embryo transfer. Lab. Anim. Sci. 38:661-2.

Critser, J. K. and L. E. Mobraaten. (2000) Cryopreservation of murine spermatozoa. ILAR J. 41:197-206.

Glenister, P. J., D. G. Whittingham and M. J. Wood. (1990) Genome cryopreservation—A valuable contribution to mammalian genetic research. Genet. Res. 56:253-258.

Glenister, P. and C. Thornton. (2000) Cryoconservation—archiving for the future. Mamm. Genome 11:565-71.

Gordon, J. W. and U. Dapunt. (1993) Restoration of normal implantation rates in mouse embryos with a hatching impairment by use of a new method of assisted hatching. Fertility and Sterility 59:1302-7.

Hogan, B. (1994) In: Manipulating the mouse embryo: A laboratory manual, Cold Spring Harbor Laboratory, Cold Spring Harbor Press, Plainview, N.Y.

Khalifa, E-A. M., M. J. Tucker and P. Hunt. (1992) Cruciate thinning of the zona pellucida for more successful enhancement of blastocyst hatching in the mouse. Hum. Repro. 7:532-6.

Kimura, Y., and R. Yanagimachi. (1995) Intracytoplasmic sperm injection in the mouse. Biol. Reprod. 52:709-20.

Lawitts, J. A. and J. D. Biggers. (1991) Optimization of mouse embryo culture media using simplex methods. J. Reprod. Fert. 91:543-556.

Mansour, R. T., C. A. Rhodes, M. A. Aboulghar, G. I. Serour and A. Kamal. (2000) Transfer of zona-free embryos improved outcome in poor prognosis patients: a prospective randomized controlled study. Hum. Repro. 15:1061-4.

Mobraaten, L. E. (1986) Mouse embryo cryobanking. J. In Vitro Fert. Emb. Transfer 56:253-8.

Mobraaten, L. E. (1998) Cryopreservation centres: a means to archive. In Novel Systems for the Study of Human Disease: From Basic Research to Applications, OECD, Editor, Organisation for Economic Co-operation and Development, Paris: Rome, Italy, p. 321-8.

Mobraaten, L. (1999) Cryopreservation in a transgenic program. Lab. Animal 33:15-18.

Morrell, J. M. (1999) Techniques of embryo transfer and facility decontamination used to improve the health and welfare of transgenic mice. Lab. Animal 33:201-6.

Naito, K., Y. Toyoda and R. Yanagimachi. (1992) Production of normal mice from oocytes fertilized and developed without zonae pellucidae. Hum. Reprod. 7:281-5.

Nakagata, N. and T. Takeshima (1992) Cryopreservation of mouse spermatozoa from inbred and F1 hybrid strains. Jikken Dobutsu 42:317-20.

Nakagata, N., M. Okamoto, O. Ueda and H. Suzuki (1997) Positive effect of partial zona-pellucida dissection on the in vitro fertilizing capacity of cryopreserved C57BL/6J transgenic mouse spermatozoa of low motility. Biol. Reprod. 57:1050-55.

Odawara, Y., I Tachibana, S. Chida, M. Hiruta, S. Iida, W. Yoko, M. Shigeru and M. Suzuki. (1993) Zona opening for severe male factor infertility. J. Fertil. Implant 10:279-281.

Odawara, Y., K. Matsumoto, S. Shinozuka, K. Kusuhara and Y. Terashima (1995) Zona manipulation in an assisted reproductive technologies (ART) programme. J. Obstet. Gynaecol. 21:83-8.

Quinn, P., Kerin, J. F. and G. M. Warnes. (1985) Improved pregnancy rate in human in vitro fertilization with the use of a medium based on the composition of human tubal fluid. Fertil. Steril. 44:493-8.

Reetz, I. C., M. Wullenweber-Schmidt, V. Kraft and H. J. Hedrich. (1988) Lab. Anim. Sci. 38:696-701.

Sharp, J. and L. Mobraaten (1997) To save or not to save: the role of repositories in a period of rapidly expanding development of genetically engineered strains of mice. In Transgenic Animals: Generation and Use, L.-M. Houdebine, Editor, Harwood Academic Publishers, Switzerland, pp. 525-32.

Sharp, J., C. Linder and L. Mobraaten. (2001) Genetically engineered mice. Husbandry and resources. Methods Mol. Biol. 38:661-2.

Suzuki, H., K. Yorozu, T. Watanabe, M. Nakura and J. Adachi. (1996) Rederivation of mice by means of in vitro fertilization and embryo transfer. Exp. Anim. 45:33-8.

Sztein, J. M., S. Farley, A. F. Young and L. E. Mobraaten. (1997) Motility of cryopreserved mouse spermatozoa affected by temperature of collection and rate of thawing. Cryobiology 35:46-52.

Sztein, J. M., J. S. Farley and L. E. Mobraaten. (2000a) In vitro fertilization with cryopreserved inbred mouse sperm. Biol. Reprod. 63:1774-80.

Sztein, J. M., M. J. O'Brien, J. S. Farley, L. E. Mobraaten and J. J. Eppig. (2000b) Rescue of oocytes from antral follicles of cryopreserved mouse ovaries: competence to undergo maturation, embryogenesis, and development to term. Hum. Reprod. 15:567-71.

Sztein, J. M., K. Noble, J. S. Farley and L. E. Mobraaten. (2001) Comparison of permeating and nonpermeating cryoprotectants for mouse sperm cryopreservation. Cryobiology 42:28-39.

Yokoyama, M., H. Akiba, M. Katsuki and T. Nomura. (1990) Production of normal young following transfer of mouse embryos obtained by in vitro fertilization using cryopreserved spermatozoa. Jikken Dobutsu 39:125-8.

Although the invention has been described with reference to the presently preferred embodiments, it should be understood that various modifications could be made without departing from the spirit of the invention.

What is claimed is:

1) A method for maintaining genetic lines in a non-human mammal comprising:
a. isolating oocytes from a donor female of said non-human mammal to produce isolated oocytes;
b. reducing the circumferential thickness of the zona pellucida layer surrounding said isolated oocytes without removing said zona pellucida layer completely;
c. fertilizing said isolated oocytes in vitro with cryogenically preserved sperm from the same species as said non-human mammal to produce at least one fertilized embryo; and
d. transplanting said fertilized embryo to a recipient female of said non-human mammal for implantation and placental development.

2) The method of claim 1, wherein said method further comprises the step of inducing said donor female to superovulate prior to said isolating.

3) The method of claim 1, wherein said reducing comprises reducing the circumferential thickness of the zona pellucida layer surrounding said isolated oocytes by approximately one-half.

4) The method of claim 1, wherein said reducing comprises treating said zona pellucida layer with acid Tyrode's solution.

5) The method of claim 1, wherein said reducing comprises treating said zona pellucida layer with an acidified salt solution.

6) The method of claim 5, wherein said acidified salt solution is chosen from the group consisting of acidified PBS, acidified HTF and acidified normal saline solution.

7) The method of claim 1, wherein said reducing comprises treating said zona pellucida layer with an enzymatic agent.

8) The method of claim 7, wherein said enzymatic agent is a proteinase which is capable of reducing said zona pellucida layer.

9) The method of claim 8, wherein said proteinase is chosen from the group consisting of pronase, hyaluronidase and trypsin.

10) The method of claim 1, wherein said method further comprises the step of cryogenically preserving said sperm with a solution consisting of raffinose and dehydrated milk prior to said fertilizing.

11) The method of claim 1, wherein said method further comprises the step of culturing said fertilized embryo to at least the 2-cell embryo stage prior to said transplanting.

12) The method of claim 1, wherein said method further comprises the step of culturing said fertilized embryo to at least the blastocyst stage prior to said transplanting.

13) The method of claim 1, wherein said non-human mammal is a mouse.

14) A method for manipulating the zona pellucida of a non-human mammalian oocyte to increase the fertilization capability of cryopreserved sperm such that the circumferential thickness of said zona pellucida surrounding said mammalian oocyte is decreased while maintaining an intact zona pellucida layer, comprising:
a. removing cumulus cells surrounding said oocyte; and
b. treating said oocyte with an agent to reduce the circumferential thickness of said zona pellucida

15) The method of claim 14, wherein said circumferential thickness of said zona pellucida is reduced by approximately one-half.

16) The method of claim 14, wherein said agent is acid Tyrode's solution.

17) The method of claim 14, wherein said agent is an acidified salt solution.

18) The method of claim 17, wherein said acidified salt solution is chosen from the group comprising acidified HTF, acidified PBS and acidified normal saline solution.

19) The method of claim 14, wherein said agent is an enzymatic agent.

20) The method of claim 19, wherein the enzymatic agent is a proteinase capable of reducing said circumferential thickness of the zona pellucida layer.

21) The method of claim 20, wherein said proteinase is chosen from the group comprising pronase, hyaluronidase and trypsin.

22) An isolated oocyte wherein the circumferential thickness of the zona pellucida layer surrounding said isolated oocyte is reduced.

23) The isolated oocyte of claim 22, wherein said circumferential thickness of the zona pellucida layer surrounding said isolated oocyte is reduced by approximately one-half.

24) The isolated oocyte of claim 22, wherein said isolated oocyte is treated with an acidified salt solution.

25) The isolated oocyte of claim 22, wherein said acidified salt solution is acid Tyrode's solution.

26) The isolated oocyte of claim 22, wherein said isolated oocyte is treated with an enzymatic agent.

27) The isolated oocyte of claim 26, wherein said enzymatic agent is a proteinase capable of reducing said circumferential thickness of the zona pellucida layer.

28) The isolated oocyte of claim 27, wherein said proteinase is chosen from the group comprising pronase, hyaluronidase and trypsin.
